(19) United States
(12) Patent Application Publication
(10) Pub. No.: US 2024/0307354 A1
(43) Pub. Date: Sep. 19, 2024

(54) METHOD FOR TREATING HEAD AND NECK CANCER

(71) Applicant: BEIJING INNOCARE PHARMA TECH CO., LTD., Beijing (CN)

(72) Inventors: Jisong CUI, Beijing (CN); Renbin ZHAO, Beijing (CN); Carrie ZHOU, Beijing (CN); Bin ZHANG, Beijing (CN)

(21) Appl. No.: 18/656,947

(22) Filed: May 7, 2024

Related U.S. Application Data
(63) Continuation of application No. PCT/CN2022/129571, filed on Nov. 3, 2022.
(60) Provisional application No. 63/279,985, filed on Nov. 16, 2021.

(51) Int. Cl. A61K 31/4155; A61P 35/00

(52) U.S. Cl. CPC A61K 31/4155; A61P 35/00

(57) ABSTRACT

The present invention relates to a method for treating head and neck cancer. The method comprises administering to a patient in need thereof an effective amount of gunagratinib, or a pharmaceutically acceptable salt thereof. The method is effective in treating squamous-cell carcinomas arising from mucosal surfaces of the oral cavity, sinonasal cavity, pharynx, larynx, and nasopharyngeal. Preferred route of administration is oral administration.

### CROSS-REFERENCE TO RELATED APPLICATION(S)

This application is a continuation of PCT/CN2022/129571, filed Nov. 3, 2022; which claims the benefit of U.S. Provisional Application No. 63/279,985, filed Nov. 16, 2021. The contents of the above-identified applications are incorporated herein by reference in their entirety.

### TECHNICAL FIELD

The present invention relates to a method for treating head and neck cancer by administering to a subject in need thereof an effective amount of gunagratinib, or a pharmaceutically acceptable salt thereof.

### BACKGROUND OF THE INVENTION

Head and neck cancer is associated with pain, disfiguration, dysfunction, psychosocial distress, and death. Head and neck cancer was the seventh most common cancer worldwide in 2018 (890,000 new cases and 450,000 deaths), accounting for 3% of all cancers and just over 1.5% of all cancer deaths in the United States (see Chow, L, N. Engl. J. Med., 382:1, 60-72, 2020).

Fibroblast Growth Factor Receptor (FGFR) belongs to the receptor tyrosine kinase (RTK) superfamily. FGFR structurally consists of an extra-membrane ligand binding domain, a single transmembrane domain, and an intra-membrane tyrosine kinase. FGFRs bind to ligands with high affinity, initiate downstream STAT3, MAPK, and PI3K/AKT signaling cascades, and are involved in several biological processes such as organogenesis, angiogenesis, cellular proliferation, migration, and anti-apoptosis. Alterations in FGFR signaling pathways play an important role in tumorigenesis and are potential therapeutic drug targets. Because FGF/FGFR pathway aberrations are cancer drivers, FGFR-targeted therapy has become an emerging area of clinical research.

FGFRs include the 4 subtypes of FGFR1, FGFR2, FGFR3, and FGFR4, which have tissue-specific expression. FGFR1 is primarily expressed in tissues of mesoderm origin, FGFR2 is expressed in tissues of endoderm origin, FGFR3 is highly expressed during neural development and in nervous tissue, and FGFR4 is distributed in liver, gallbladder, lung, kidney, and muscle tissues and is highly expressed during endoderm development. Abnormal FGF/FGFR activation occurs in several solid and blood cancers and has been identified as a driver of cholangiocarcinoma and bladder, endometrial, breast, stomach, lung, and ovarian cancers.

A variety of tumors are closely related to FGF/FGFR expression and activation, such as non-small cell lung cancer, breast cancer, gastric cancer, liver cancer, bladder cancer, endometrial cancer, prostate cancer, cervical cancer, colon cancer, esophageal cancer, myeloma and melanoma, and so on (Clin. Cancer Res. 2012, 18, 1855). Studies have shown that FGFR1 amplification accounts for 20% of non-small cell lung cancer, FGFR2 amplification accounts for about 5% of gastric cancer, FGFR3 mutation accounts for about 70% of non-invasive bladder cancer, and FGFR4 is amplified in liver cancer (PloS One 2012, 7, e36713). Therefore, the development of inhibitors targeting FGFR has become a hot topic in anti-tumor drug research (Drug Disc. Today 2014, 19, 51).

### BRIEF DESCRIPTION OF THE DRAWINGS

FIG. 1 shows the results of 7 patients with head and neck cancers treated with gunagratinib for at least 2 cycles. Each cycle is 21 days.

### DETAILED DESCRIPTION OF THE INVENTION

### Definitions

“Head and neck cancer” refers to squamous-cell carcinomas arising from mucosal surfaces of major anatomical sites: the oral cavity, sinonasal cavity, pharynx, larynx, and nasopharyngeal. The oral cavity includes the lips, buccal mucosa, anterior tongue, floor of the mouth, hard palate, upper and lower gingiva, and retromolar trigone. The pharynx includes the nasopharynx (behind the nasal cavity), oropharynx (comprising the tonsillar area, tongue base, soft palate, and posterior pharyngeal wall), and hypopharynx (comprising the pyriform sinuses, posterior surface of the larynx and postcricoid area, and inferior posterior and inferolateral pharyngeal walls). The larynx includes the supraglottic larynx, glottic larynx (true vocal cords and anterior and posterior commissures), and subglottic larynx. The nasal cavity and paranasal sinuses include the maxillary, ethmoid, sphenoid, and frontal sinuses.

“Isomer” refers to a compound having the same molecular formula but differing in the nature or sequence of its atomic bonding or in the spatial arrangement of its atoms, which is referred to as an “isomer.” An isomer whose atomic space is arranged differently are referred to as a “stereoisomer.” Stereoisomers include optical isomers, geometric isomers, and conformational isomers.

“Isotopes” include those atoms having the same atomic number but different mass numbers. Examples of isotopes suitable for incorporation into the compounds of the present invention are hydrogen, carbon, nitrogen, oxygen, phosphorus, fluorine and chlorine, for example but not limited to ²H, ³H, ¹³C, ¹⁴C, ¹⁵N, ¹⁸O, ¹⁷P, ³¹P, ³²P, ³⁵S, ¹⁸F and ³⁶Cl. Isotopically labeled compounds of the present invention can generally be prepared by conventional technical means known to a person of ordinary skill in the art or by methods analogous to those described in the accompanying examples, using the appropriate isotopically labeled reagents in place of the non-isotopically labeled reagents. Stable isotopes such as 2H (deuterium) may advantageously alter biological, pharmacological or pharmacokinetic properties.

“Prodrug” refers to derivative of gunagratinib which can be converted to the biologically active compound under a physiological condition in vivo, for example by oxidation, reduction, hydrolysis, and so on, each of which is carried out using an enzyme or without the participation of an enzyme. An example of a prodrug is a compound in which an amino group in a compound of the invention is acylated, alkylated or phosphorylated, such as eicosylamino, alanylamino, pivaloyloxymethylamino, or wherein the hydroxy group is acylated, alkylated, phosphorylated or converted to a borate, for example acetoxy, palmitoyloxy, pivaloyloxy, succinyloxy, fumaryloxy, alanyloxy, and the like, or a carrier molecule in which the carboxyl group is esterified or amidated, or wherein the thiol group forms a disulfide bridge with a group selectively delivers a drug to the target and/or to the cytosol of the cell, such as a peptide. These compounds can be prepared from the compounds of the present invention according to certain known methods.

“Pharmaceutically acceptable salt” refers to a salt made from a pharmaceutically acceptable base or acid, including inorganic bases or acids and organic bases or acids. For example, pharmaceutically acceptable salt includes an alkali metal salt, an alkaline earth metal salt or as an ammonium salt. More specific examples of such salts include sodium salts, potassium salts, calcium salts, magnesium salts, or addition salts with inorganic or organic acids. Examples of suitable acids include hydrochloric acid, hydrobromic acid, phosphoric acid, sulfuric acid, nitric acid, methanesulfonic acid, p-toluenesulfonic acid, naphthalenedisulfonic acid, oxalic acid, acetic acid, tartaric acid, lactic acid, salicylic acid, benzoic acid, formic acid, propionic acid, pivalic acid, malonic acid, succinic acid, pimelic acid, fumaric acid, maleic acid, malic acid, sulfamic acid, phenylpropionic acid, gluconic acid, ascorbic acid, isonicotinic acid, citric acid, adipic acid and other acids known to a person of ordinary skill in the art.

The present invention provides a method for treating head and neck cancer. The method comprises administering to a patient in need thereof an effective amount of gunagratinib, or a prodrug thereof, a stable isotope derivative thereof, a pharmaceutically acceptable salt thereof, a stereoisomer thereof, or a mixture thereof.

Gunagratinib is an INN (International Nonproprietary Names) name; its chemical name is (S)-1-(1-acryloylpyrrolidin-3-yl)-3-((3,5-dimethoxyphenyl)ethynyl)-5-(methylamino)-1H-pyrazole-4-carboxamide, and its structure is shown below

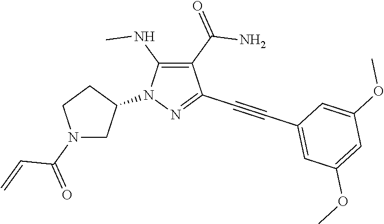 [SMILES: C=CC(=O)N1CC[C@H](n2nc(C#Cc3cc(OC)cc(OC)c3)c(C(N)=O)c2NC)C1]

An effective amount refers to an amount of gunagratinib that is effective to inhibit the function of FGFR and treat the head and neck cancer.

### Pharmaceutical Compositions

The present invention provides pharmaceutical compositions comprising one or more pharmaceutically acceptable carriers and an active compound of gunagratinib. The active compound in the pharmaceutical compositions in general is in an amount of about 0.1-5% for an injectable formulation, about 1-90% for a tablet formulation, and 1-100% for a capsule formulation.

In one embodiment, the pharmaceutical composition can be in a dosage form such as tablets, capsules, granules, fine granules, powders, syrups, suppositories, injectable solutions, patches, or the like. The pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, and gels. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as microcrystalline cellulose, methylcellulose, hydroxypropyl cellulose, hydroxyethyl cellulose, carboxymethyl cellulose, and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate; xanthan gum, and carrageenan. Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the active compound may contain other excipients that have no bioactivity and no reaction with the active compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of excipients of a tablet or a capsule include, but not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone. For example, a tablet formulation may contain inactive ingredients such as colloidal silicon dioxide, crospovidone, hypromellose, magnesium stearate, microcrystalline cellulose, polyethylene glycol, sodium starch glycolate, and/or titanium dioxide. A capsule formulation may contain inactive ingredients such as gelatin, magnesium stearate, and/or titanium dioxide.

### Method of Use

The present invention is directed to a method of head and neck cancer in a patient. Gunagratinib can be administered in the form of a pharmaceutical composition that additionally contains a pharmaceutically acceptable carrier. The present method comprises administering to a patient in need thereof an effective amount of gunagratinib, a prodrug thereof, a stable isotope derivative thereof, a pharmaceutically acceptable salt thereof, a stereoisomer thereof, or a mixture thereof.

The method comprises the steps of first identifying a subject suffering from head and neck cancer, and administering to the subject an effective amount of gunagratinib.

The method is effective in treating squamous-cell carcinomas arising from mucosal surfaces of the oral cavity, sinonasal cavity, pharynx, larynx, and nasopharyngeal.

The method treats head and neck cancer caused by FGF/FGFR signaling pathway aberration, including but not limited to gene mutations, amplifications, rearrangements/fusions, insertion/deletions, and protein over-expression. For example, the method treats FGF3, FGF4, and/or FGF19 amplifications, and/or FGFR (e.g., FGFR 1/2/3/4) overexpression.

The present invention provides use of gunagratinib, a prodrug thereof, a stable isotope derivative thereof, a pharmaceutically acceptable salt thereof, a stereoisomer thereof, or a mixture thereof, for preparing a medicament for treating head and neck cancer in a patient.

The present invention provides gunagratinib, a prodrug thereof, a stable isotope derivative thereof, a pharmaceutically acceptable salt thereof, a stereoisomer thereof, or a mixture thereof, for use in a method of treating head and neck cancer in a patient.

The pharmaceutical composition of the present invention can be applied by local administration and systemic administration. Local administration includes topical administration. Systemic administration includes oral, parenteral (such as intravenous, intramuscular, subcutaneous or rectal), and other systemic routes of administration. In systemic administration, the active compound first reaches plasma and then distributes into target tissues. Oral administration is a preferred route of administration for the present invention.

Dosing of the composition can vary based on the extent of the injury and each patient's individual response, and the possibility of co-usage with other therapeutic treatments including use of other therapeutic agents.

In general, an effective dose of gunagratinib to be orally administered to a human subject is about 5-50 mg per day, preferably 10-30 mg per day. The daily dosage may be administered in one administration or in separate administrations of 2 to 4 doses such as equal doses.

In one embodiment, the pharmaceutical composition is administrated subcutaneously to the subject.

The time period for which the subject is dosed with gunagratinib in any of the methods described above can range, for example, from about 1 to 2 weeks to the remaining lifespan of the subject. Gunagratinib can be dosed, for example, for at least 1, 2, 3, 4, 5, 6, 7, 8, 9, 10, 20, 30, 40, or 50 weeks, or at least 1, 2, 3, 4, 5, 6, 7, 8, 9, 11, or 12 months, or at least 1 to 2 years.

Those of skill in the art will recognize that a wide variety of delivery mechanisms are also suitable for the present invention.

The present invention is useful in treating a mammal subject, such as humans, horses, and dogs. The present invention is particularly useful in treating humans.

The following examples further illustrate the present invention. These examples are intended merely to be illustrative of the present invention and are not to be construed as being limiting.

### EXAMPLE

### Example 1. Head and Neck Cancer Patients Treated with Gunagratinib

### Objectives

The study is to evaluate efficacy of orally administered gunagratinib for treating head and neck cancer in patient.

### Main Criteria for Inclusion:

Participants are eligible to be included in the study only if all of the following criteria apply:
1. Patients with histologically confirmed unresectable or metastatic advanced malignant solid tumors, who are refractory or relapsed, or are intolerant to all standard treatment regimens or have no standard treatment available (dose escalation part).
2. Patients with histologically or cytologically confirmed unresectable, relapsed, or metastatic cholangiocarcinoma (AJCC <2017 version 8>TNM stage IV), or who are intolerant to first-line chemotherapy (at least two cycles of chemotherapy are required), or who have progressed/relapsed within 6 months after neoadjuvant/adjuvant chemotherapy (dose expansion part).
3. Existing test reports have confirmed the FGFR2 gene translocation/fusion or the central laboratory has detected the FGFR2 gene translocation/fusion (dose expansion part).
4. Age ≥18 and ≤75 years old.
5. At least one evaluable lesion (dose escalation part) and at least one measurable lesion (dose expansion part), according to the Response Evaluation Criteria in Solid Tumors, version 1.1 (RECIST 1.1).
6. ECOG performance status of 0-1 (dose escalation part); ECOG performance status of 0-2 (dose expansion part).
7. Life expectancy of more than 3 months.
8. Must have adequate organ function as described below:
   a) Bone marrow: Absolute neutrophil count (ANC) ≥1.5×10⁹/L (1500/mm³), platelets ≥75×10⁹/L, hemoglobin ≥90 g/L (9 g/dL).
   b) Coagulation: Prothrombin time (PT), international normalized ratio (INR), and activated partial thromboplastin time (APTT) are all ≤1.5×the upper limits of normal (ULN).
   c) Liver: Serum bilirubin ≤1.5×ULN (≤2.5×ULN if the tumor metastasizes the liver), aspartate aminotransferase (AST) and alanine aminotransferase (ALT) are ≤3×ULN (if the tumor metastasizes the liver, AST and ALT≤5×ULN).
   d) Serum creatinine <1.5×ULN or creatinine clearance >60 mL/min (calculated according to Cockcroft-Gault formula).
9. Participate voluntarily, sign informed consent, and follow the study treatment plan and scheduled visits.

### Main Criteria for Exclusion:

Participants are excluded from the study if any of the following criteria apply:
1. Have previously been treated with selective pan-FGFR molecular inhibitors or antibody drugs (such as erdafitinib, pemigatinib, infrigatinib, rogaratinib, derazantinib, HMPL-453, 3D185, ECO317, E7090, etc.).
2. Received oral fluorouracil chemotherapy within 2 weeks before taking the investigational drug for the first time and received systemic or local anti-cancer treatment within 4 weeks, including chemotherapy (except oral fluorouracil chemotherapy), radiotherapy, hormone or targeted drugs and biological.
3. Major surgery (thoracotomy, laparotomy, etc.) within 6 weeks or minor surgery (superficial skin surgery, lymphadenectomy, hernia repair, etc.) within 2 weeks before the first dose of the study drug.
4. Within 2 weeks before the first dose of the study drug, the patient's phosphate level continues to exceed the ULN despite medical treatment.
5. Patients with clinically significant gastrointestinal dysfunction that may affect drug intake, transport, or absorption (e.g., inability to swallow, chronic diarrhea, intestinal obstruction, etc.).
6. Has known symptomatic central nervous system metastases. No symptomatic central nervous system metastases or stable disease ≥4 weeks after treatment is eligible.
7. Has a history of or currently uncontrolled cardiovascular diseases including:
   Known New York Heart Association (NYHA) Grade II or higher congestive heart failure, unstable angina pectoris, or myocardial infarction within 6 months before the first dose of the study drug.
   Arrhythmias requiring treatment at screening or left ventricular ejection fraction (LVEF) <50% at screening.
   Clinically significant prolonged QTc interval, or QTc interval >470 ms in women and >450 ms in men at screening.
8. History of active bleeding within 6 months, signs of portal hypertension leading to gastric esophageal venous bleeding within 2 months before the first dose of the study drug, or the investigator believes that there is a clear bleeding tendency (such as esophageal varices, local active ulcer lesions, etc.).
9. At the investigator's discretion, evidence of severe or uncontrolled systemic disease (such as unstable or non-compensatory lung, liver, or kidney disease); or any unstable systemic disease (including active clinically severe infections, uncontrolled hypertension, or liver, kidney, or metabolic disease).
10. History of interstitial pulmonary disease, pulmonary embolism, and deep venous embolism (phase I: dose escalation).
11. History of stroke or intracranial hemorrhage within 6 months prior to taking the investigational drug.
12. History of organ transplantation or a history of allogeneic hematopoietic stem cell transplantation (phase I: dose escalation).
13. Current evidence of corneal or retinal abnormalities that may increase ocular toxicity, including but not limited to:
   a) Currently suffering from central serous retinopathy (CSR) or retinal vein occlusion (RVO), or with relevant history.
   b) Active wet and senile macular degeneration (AMD).
   c) Diabetic retinopathy with macular edema.
   d) Uncontrollable glaucoma.
   e) Keratopathy, such as keratitis, keratoconjunctivitis, keratopathy, corneal abrasion, inflammation, or ulceration.
14. Active hepatitis B virus (HBV DNA >ULN tested by PCR), active hepatitis C, or HIV infection.
15. Has not recovered from reversible toxicity (>Grade 1) of prior therapy (except alopecia, controllable nausea and vomiting).
16. Pregnant or lactating women, as well as women with childbearing potential who are unwilling or unable to perform contraception from screening to 6 months after the last study drug administration; and fertile men who are unwilling or unable to perform contraception from screening to 12 weeks after the last study drug administration.
17. Other conditions considered by the investigator to be inappropriate for participation in this study.

### Dose and Mode of Administration:

Patients are treated with 14 mg, 16 mg, 18 mg, or 22 mg of gunagratinib once a day per 21-day cycle. Patients are evaluated on Day 21 every two cycles.

### Methods of Evaluation

### Eastern Cooperative Oncology Group (ECOG) Performance Status

ECOG Performance Status is a simple measure of functional status of a cancer patient.
Score 0: Fully active, able to carry on all pre-disease performance without restriction
Score 1: Restricted in physically strenuous activity but ambulatory and able to carry out work of a light or sedentary nature, e.g., light housework, office work
Score 2: Ambulatory and capable of all self-care but unable to carry out any work activities. Up and about more than 50% of waking hours
Score 3: Capable of only limited self-care, confined to bed or chair more than 50% of waking hours
Score 4: Completely disabled. Cannot carry on any self-care. Totally confined to bed or chair
Score 5: Dead
   Response Evaluation Criteria in Solid tumor.

At baseline, tumor lesions/lymph nodes is categorized measurable or non-measurable as follows:

### Measurable

Tumor lesions: Must be accurately measured in at least one dimension (longest diameter in the plane of measurement is to be recorded) with a minimum size of:
10 mm by CT scan (CT scan slice thickness no greater than 5 mm; see Appendix II on imaging guidance).
10 mm caliper measurement by clinical exam (lesions which cannot be accurately measured with calipers should be recorded as non-measurable).
20 mm by chest X-ray

Malignant lymph nodes: To be considered pathologically enlarged and measurable, a lymph node must be P15 mm in short axis when assessed by CT scan (CT scan slice thickness recommended to be no greater than 5 mm). At baseline and in follow-up, only the short axis will be measured and followed. See also notes below on ‘Baseline documentation of target and non-target lesions’ for information on lymph node measurement.

### Non-Measurable

All other lesions, including small lesions (longest diameter <10 mm or pathological lymph nodes with P10 to <15 mm short axis) as well as truly non-measurable lesions. Lesions considered truly non-measurable including leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

### Method of Assessment

Clinical lesions: Clinical lesions is only considered measurable when they are superficial and P10 mm diameter as assessed using calipers (e.g., skin nodules). For the case of skin lesions, documentation by color photography including a ruler to estimate the size of the lesion is suggested. When lesions can be evaluated by both clinical exam and imaging, imaging evaluation should be undertaken.

CT, MRI: CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable in certain situations (e.g., for body scans).

### Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

Stable Disease (SD): Neither sufficient shrinkage (compared to baseline) to qualify for PR nor sufficient increase (taking as reference the smallest sum diameters while on study) to qualify for PD.

The objective response rate is calculated by number of patients having complete response (CR) and partial response (PD) divided by total number of patients.

The disease control rate (DCR) is calculated by number of patients having complete response (CR), partial response (PR), and stable disease (SD), divided by total number of patients.

### Head and Neck Patients

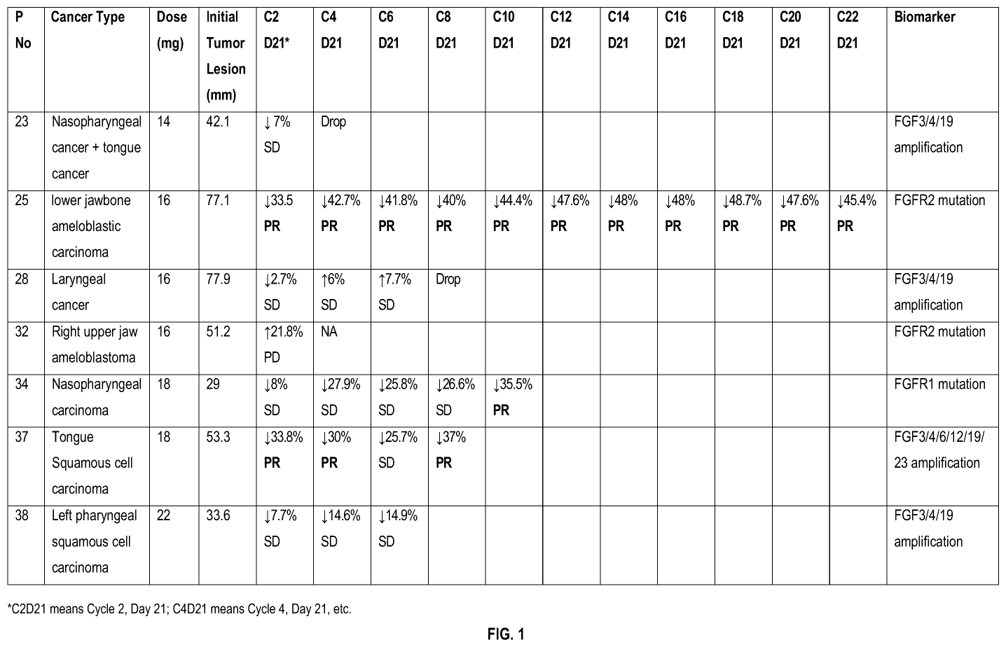

7 patients with head and neck cancers have been treated for at least 2 cycles. The results are shown in FIG. 1.

Patient Nos. 23, 28, 38 have FGF 3, 4, 19 ligand amplification. Patient No. 25 has a mutation C383R in FGFR2. Patient No. 32 has a mutation R450H in FGFR2. Patient No. 34 has FGFR1-P150S subcloning. Patient No. 37 has FGF3/4/6/12/19/23 amplification.

### Results

The results of 7 patients are shown in FIG. 1. The treatments of Patient Nos. 25, 34, 37, and 38 are still on-going.

Patient 25 consistently shows a partial response (PR) of a decrease of cancer lesion of over 42% after 4-22 cycles of treatment.

Patient 37 shows PR of a decrease of cancer lesion of 33.8%, 30%, and 37% after 2, 4, and 8 cycles of treatment, respectively.

Patient 34 shows stable disease (SD) after 2-8 cycles of treatment, and shows PR after 10 cycles of treatment.

Three Patients (23, 28, and 38) show stable disease (SD) after treatment.

Patient 32 shows progressive disease (PD).

It is to be understood that the foregoing describes preferred embodiments of the present invention and that modifications may be made therein without departing from the scope of the present invention as set forth in the claims.

What is claimed is:

1. A method for treating head and neck cancer, comprising administering to a patient in need thereof an effective amount of gunagratinib, a prodrug thereof, a stable isotope derivative thereof, a pharmaceutically acceptable salt thereof, a stereoisomer thereof, or a mixture thereof.

2. The method of claim 1, wherein gunagratinib is administered orally.

3. The method of claim 2, wherein gunagratinib is administered at a daily dosage of 5-50 mg to the patient.

4. The method of claim 3, wherein gunagratinib is administered at a daily dosage of 10-30 mg to the patient.

5. The method of claim 1, which reduces the size of cancer lesion in the patient.
